(19) United States
(12) Patent Application Publication
(10) Pub. No.: US 2025/0271343 A1
(43) Pub. Date: Aug. 28, 2025

(54) METHOD AND SYSTEM FOR OBTAINING PYROLYSIS KINETICS PARAMETER OF SOLID MATERIAL AND STORAGE MEDIUM

(71) Applicant: SHANGHAI TOSUN TECHNOLOGY LTD., Shanghai (CN)

(72) Inventors: Mang MO, Shanghai (CN); Jinpeng XU, Shanghai (CN); Chu LIU, Shanghai (CN); Yueyin XIE, Shanghai (CN)

(73) Assignee: SHANGHAI TOSUN TECHNOLOGY LTD., Shanghai (CN)

(21) Appl. No.: 18/757,776

(22) Filed: Jun. 28, 2024

(30) Foreign Application Priority Data
Feb. 23, 2024 (CN) 202410203886.3

(51) Int. Cl. G01N 5/04; G01N 1/44

(52) U.S. Cl. CPC G01N 5/04; G01N 1/44

(57) ABSTRACT

A method and a system for obtaining a pyrolysis kinetics parameter of a solid material and a storage medium are provided. The method includes: collecting an experimental curve of a mass loss percentage of a pyrolysate sample along with change of temperature or time; constructing a kinetics mechanism function library and traversing each kinetics mechanism function to obtain a simulation curve of the mass loss percentage corresponding to each kinetics mechanism function along with change of temperature or time; respectively calculating a root mean square error (RMSE) of the simulation curve of each mass loss percentage along with change of temperature or time and the experimental curve of the mass loss percentage along with change of temperature or time; and, sorting each RMSE and taking the kinetics mechanism function corresponding to a minimum RMSE as the pyrolysis kinetics parameter of the solid material.

### CROSS REFERENCE TO THE RELATED APPLICATIONS

This application is based upon and claims priority to Chinese Patent Application No. 202410203886.3, filed on Feb. 23, 2024, the entire contents of which are incorporated herein by reference.

### TECHNICAL FIELD

The present disclosure relates to the field of analysis technologies of pyrolysis kinetics parameters and in particular to a method and system for obtaining a pyrolysis kinetics parameter of a solid material and a storage medium.

### BACKGROUND

Pyrolysis kinetics is a branch subject in which physical changes or chemical reaction kinetics is researched by thermal analysis or calorimetric technology. In the pyrolysis kinetics research, the most probable mechanism function is one of the important pyrolysis kinetics parameters of solid materials.

In the related arts, the most probable mechanism function is mostly obtained by subjective inference or approximate inference. In this case, the obtained most probable mechanism function is usually inaccurate and thus needs to be verified repeatedly, affecting the obtaining efficiency of the pyrolysis kinetics parameters of solid materials.

### SUMMARY

The object of the present disclosure is to provide a method and system for obtaining a pyrolysis kinetics parameter of a solid material, and a storage medium.

In order to solve the above technical problems, the present disclosure provides a method of obtaining a pyrolysis kinetics parameter of a solid material. The method includes:
collecting an experimental curve of a mass loss percentage of a pyrolysate sample along with change of temperature or time;
constructing a kinetics mechanism function library and traversing each kinetics mechanism function to obtain a simulation curve of the mass loss percentage corresponding to each kinetics mechanism function along with change of temperature or time;
respectively calculating a root mean square error (RMSE) of the simulation curve of each mass loss percentage along with change of temperature or time and the experimental curve of the mass loss percentage along with change of temperature or time; and,
sorting each RMSE and taking the kinetics mechanism function corresponding to a minimum RMSE as the pyrolysis kinetics parameter of the solid material.

The present disclosure has the following beneficial effects: in the method of obtaining the pyrolysis kinetics parameter of the solid material, each kinetics mechanism function is traversed sequentially based on function sequence number and in this traversal process, based on quasi-newton method, the kinetics parameters the activation energy E and the pre-exponential factor A of each kinetics mechanism function are optimized to reduce an error between the simulation curve of each mass loss percentage along with change of temperature or time and the experimental curve of the mass loss percentage along with change of temperature or time and thus each of the RMSE values is already optimized to the minimum so as to approach a theoretical minimum value, ensuring the accuracy of the sorting of the kinetics mechanism functions. Furthermore, since all kinetics mechanism functions in the function library are traversed sequentially, the global property of the most probable mechanism function is ensured and the most probable mechanism function obtained hereby has a high accuracy, thereby improving the obtaining efficiency of the pyrolysis kinetics parameter of the solid material.

### BRIEF DESCRIPTION OF THE DRAWINGS

FIG. 1 is a step diagram of a method of obtaining a pyrolysis kinetics parameter of a solid material according to some embodiments of the present disclosure.

FIG. 2 is a comparison diagram of No. 3 most probable mechanism function and experimental data in cases involved in some embodiments of the present disclosure.

FIG. 3 is a comparison diagram of No. 10 non-most probable mechanism function and experimental data in cases involved in some embodiments of the present disclosure.

FIG. 4 is a principle block diagram of a system for obtaining a pyrolysis kinetics parameter of a solid material according to some embodiments of the present disclosure.

FIG. 5 is a principle block diagram of a storage medium according to some embodiments of the present disclosure.

### DETAILED DESCRIPTION OF THE EMBODIMENTS

In the related arts, the most probable mechanism function in the pyrolysis kinetics parameters of the solid materials is mostly obtained by subjective inference or approximate inference. In this case, the obtained most probable mechanism function is usually inaccurate and thus needs to be verified repeatedly, affecting the obtaining efficiency of the pyrolysis kinetics parameters of solid materials.

Therefore, at least one embodiment provides a method of obtaining a pyrolysis kinetics parameter of a solid material. The method includes the following steps:
collecting an experimental curve of a mass loss percentage of a pyrolysate sample along with change of temperature or time;
constructing a kinetics mechanism function library and traversing each kinetics mechanism function to obtain a simulation curve of the mass loss percentage corresponding to each kinetics mechanism function along with change of temperature or time;
respectively calculating a root mean square error (RMSE) of the simulation curve of each mass loss percentage along with change of temperature or time and the experimental curve of the mass loss percentage along with change of temperature or time; and,
sorting each RMSE and taking the kinetics mechanism function corresponding to a minimum RMSE as the pyrolysis kinetics parameter of the solid material.

Various non-limiting implementations of the embodiments of the present disclosure will be described in details below in combination with the drawings.

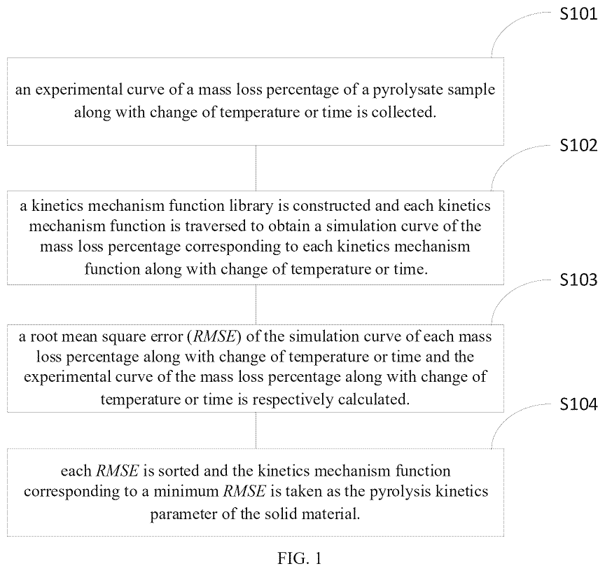

As shown in FIG. 1, some embodiments provide a method of obtaining a pyrolysis kinetics parameter of a solid material, and the method includes the following steps.

At step S101, an experimental curve of a mass loss percentage of a pyrolysate sample along with change of temperature or time is collected.

At step S102, a kinetics mechanism function library is constructed and each kinetics mechanism function is traversed to obtain a simulation curve of the mass loss percentage corresponding to each kinetics mechanism function along with change of temperature or time.

At step S103, a root mean square error (RMSE) of the simulation curve of each mass loss percentage along with change of temperature or time and the experimental curve of the mass loss percentage along with change of temperature or time is respectively calculated.

At step S104, each RMSE is sorted and the kinetics mechanism function corresponding to a minimum RMSE is taken as the pyrolysis kinetics parameter of the solid material.

Specifically, at least one embodiment includes but not limited to a library containing 41 common kinetics mechanism functions as shown in Table 1 below, where a refers to a conversion rate and namely, to a conversion percentage or fraction of a reactant; and G(α) and f(α) are mechanism functions in the form of integral and differential respectively.

TABLE 1
Common kinetics mechanism function library

| Function sequence | | |
|---|---|---|
| number | G(α) | f(α) |
| 1 | α² | $\frac{1}{2} {α}^{- 1}$ |
| 2 | α + (1 − α)ln(1 − α) | [−ln(1 − α)]⁻¹ |
| 3 | ${[ 1 - {( 1 - α )}^{\frac{1}{2}} ]}^{\frac{1}{2}}$ | $4 {{( 1 - α )}^{\frac{1}{2}} [ 1 - {( 1 - α )}^{\frac{1}{2}} ]}^{\frac{1}{2}}$ |
| 4 | ${[ 1 - {( 1 - α )}^{\frac{1}{2}} ]}^{2}$ | ${{( 1 - α )}^{\frac{1}{2}} [ 1 - {( 1 - α )}^{\frac{1}{2}} ]}^{- 1}$ |
| 5 | ${[ 1 - {( 1 - α )}^{\frac{1}{3}} ]}^{\frac{1}{2}}$ | $6 {{( 1 - α )}^{\frac{2}{3}} [ 1 - {( 1 - α )}^{\frac{1}{3}} ]}^{\frac{1}{2}}$ |
| 6 | ${[ 1 - {( 1 - α )}^{\frac{1}{3}} ]}^{2}$ | $\frac{3}{2} {{( 1 - α )}^{\frac{2}{3}} [ 1 - {( 1 - α )}^{\frac{1}{3}} ]}^{- 1}$ |
| 7 | $1 - \frac{2}{3} α - {( 1 - α )}^{\frac{2}{3}}$ | ${\frac{3}{2} [ {( 1 - α )}^{- \frac{1}{3}} - 1 ]}^{- 1}$ |
| 8 | ${[ {( 1 + α )}^{\frac{1}{3}} - 1 ]}^{2}$ | $\frac{3}{2} {{( 1 + α )}^{\frac{2}{3}} [ {( 1 - α )}^{\frac{1}{3}} - 1 ]}^{- 1}$ |
| 9 | ${[ {( 1 - α )}^{- \frac{1}{3}} - 1 ]}^{2}$ | $\frac{3}{2} {{( 1 - α )}^{\frac{4}{3}} [ {( 1 - α )}^{- \frac{1}{3}} - 1 ]}^{- 1}$ |
| 10 | ${[ - ln ( 1 - α ) ]}^{\frac{1}{4}}$ | $4 {( 1 - α ) [ - ln ( 1 - α ) ]}^{\frac{3}{4}}$ |
| 11 | ${[ - ln ( 1 - α ) ]}^{\frac{1}{3}}$ | $3 {( 1 - α ) [ - ln ( 1 - α ) ]}^{\frac{2}{3}}$ |
| 12 | ${[ - ln ( 1 - α ) ]}^{\frac{2}{5}}$ | $\frac{5}{2} {( 1 - α ) [ - ln ( 1 - α ) ]}^{\frac{3}{5}}$ |
| 13 | ${[ - ln ( 1 - α ) ]}^{\frac{1}{2}}$ | $2 {( 1 - α ) [ - ln ( 1 - α ) ]}^{\frac{1}{2}}$ |
| 14 | ${[ - ln ( 1 - α ) ]}^{\frac{2}{3}}$ | $\frac{3}{2} {( 1 - α ) [ - ln ( 1 - α ) ]}^{\frac{1}{3}}$ |
| 15 | ${[ - ln ( 1 - α ) ]}^{\frac{3}{4}}$ | $\frac{4}{3} {( 1 - α ) [ - ln ( 1 - α ) ]}^{\frac{1}{4}}$ |
| 16 | −ln(1 − α) | 1 − α |
| 17 | ${[ - ln ( 1 - α ) ]}^{\frac{3}{2}}$ | $\frac{2}{3} {( 1 - α ) [ - ln ( 1 - α ) ]}^{\frac{1}{2}}$ |
| 18 | [−ln(1 − α)]² | $\frac{1}{2} {( 1 - α ) [ - ln ( 1 - α ) ]}^{- 1}$ |
| 19 | [−ln(1 − α)]³ | $\frac{1}{3} {( 1 - α ) [ - ln ( 1 - α ) ]}^{- 2}$ |
| 20 | [−ln(1 − α)]⁴ | $\frac{1}{4} {( 1 - α ) [ - ln ( 1 - α ) ]}^{- 3}$ |
| 21 | $ln ( \frac{α}{1 - α} )$ | α(1 − α) |
| 22 | α^{1/4} | 4α^{3/4} |
| 23 | α^{1/3} | 3α^{2/3} |
| 24 | α^{1/2} | 2α^{1/3} |
| 25 | α | 1 |
| 26 | α^{3/2} | $\frac{2}{3} {α}^{- \frac{1}{2}}$ |
| 27 | α² | $\frac{1}{2} {α}^{- 1}$ |
| 28 | $1 - {( 1 - α )}^{\frac{1}{4}}$ | $4 {( 1 - α )}^{\frac{3}{4}}$ |
| 29 | $1 - {( 1 - α )}^{\frac{1}{3}}$ | $3 {( 1 - α )}^{\frac{2}{3}}$ |
| 30 | $3 [ 1 - {( 1 - α )}^{\frac{1}{3}} ]$ | ${( 1 - α )}^{\frac{2}{3}}$ |
| 31 | $1 - {( 1 - α )}^{\frac{1}{2}}$ | $2 {( 1 - α )}^{\frac{1}{2}}$ |
| 32 | $2 [ 1 - {( 1 - α )}^{\frac{1}{2}} ]$ | ${( 1 - α )}^{\frac{1}{3}}$ |
| 33 | 1 − (1 − α)² | ${[ - ln ( 1 - α ) ]}^{\frac{1}{4}}$ |
| 34 | 1 − (1 − α)³ | $\frac{1}{3} {( 1 - α )}^{- 2}$ |
| 35 | 1 − (1 − α)⁴ | $\frac{1}{4} {( 1 - α )}^{- 3}$ |
| 36 | (1 − α)⁻¹ | (1 − α)² |
| 37 | (1 − α)⁻¹ − 1 | (1 − α)² |
| 38 | ${( 1 - α )}^{- \frac{1}{2}}$ | $2 {( 1 - α )}^{\frac{3}{2}}$ |
| 39 | lnα | α |
| 40 | lnα² | ½α |
| 41 | (1 − α)⁻² | $\frac{1}{2} ( 1 - α ) 3$ |

In some embodiments, a method of traversing each kinetics mechanism function to obtain the simulation curve of the mass loss percentage corresponding to each kinetics mechanism function along with change of temperature or time includes:
setting an initial activation energy E and an initial pre-exponential factor A;
sequentially traversing each kinetics mechanism function and based on quasi-newton method, optimizing the initial activation energy E and the initial pre-exponential factor A at the time of the traversal to obtain the optimized activation energy E and pre-exponential factor A corresponding to each kinetics mechanism function; wherein the optimization formula is:

$$( {E}_{a + 1} , {A}_{a + 1} ) = ( {E}_{a} , {A}_{a} ) - {Hessian ( RMSE ( {E}_{a} , {A}_{a} ) )}^{- 1} ∇ RMSE ( {E}_{a} , {A}_{a} )$$

with each kinetics mechanism function as thermogravimetric simulation model, using the optimized activation energy E and pre-exponential factor A as parameters of each thermogravimetric simulation model to perform simulation and outputting the simulation curve of the mass loss percentage corresponding to each kinetics mechanism function along with change of temperature or time; wherein a is an iteration step number in the optimization process and a≥1.

Specifically, the initial activation energy E and the initial pre-exponential factor A are each assigned an initial value by human, and for example, the initial value of E is 70000 and the initial value of the A is e\hat{ }10, with an error of ±10%.

In the method of obtaining the pyrolysis kinetics parameter of the solid material in this embodiment, each kinetics mechanism function is traversed sequentially based on function sequence number and in this traversal process, based on quasi-newton method, the kinetics parameters the activation energy E and the pre-exponential factor A are optimized, and then with each kinetics mechanism function as simulation model, the optimized activation energy E and pre-exponential factor A are used as the parameters of the simulation model to obtain the simulation curve of the mass loss percentage along with change of temperature or time and then with the RMSE of the experimental curve of the mass loss percentage along with change of temperature or time and the simulation curve of the mass loss percentage along with change of temperature or time as target function, 41 RMSE values are obtained; then, the RMSE values are sorted and the kinetics mechanism functionvalue corresponding to the minimum RMSE value is the most probable mechanism function. Since the parameters the activation energy E and the pre-exponential factor A of the simulation model of each kinetics mechanism function are already optimized, an error between the simulation curve of each mass loss percentage along with change of temperature or time and the experimental curve of the mass loss percentage along with change of temperature or time is reduced and thus each of the RMSE values is already optimized to the minimum so as to approach a theoretical minimum value, ensuring the accuracy of the sorting of the kinetics mechanism functions. Furthermore, since all kinetics mechanism functions in the function library are traversed sequentially, the global property of the most probable mechanism function is ensured, and the most probable mechanism function obtained hereby has a high accuracy, thereby improving the obtaining efficiency of the pyrolysis kinetics parameter of the solid material.

Finally, the most probable mechanism function, the kinetics parameter the activation energy E of the most probable mechanism function, the kinetics parameter the pre-exponential factor A of the most probable mechanism function and the RMSE with the experimental curve of the mass loss percentage along with change of temperature or time are output as the pyrolysis kinetics parameters of the solid material. The more accurate kinetics mechanism functions and kinetics parameters can help construct a more accurate application model for the industrial processes such as pyrolysis, gasification and combustion and the like and thus the internal working conditions of the reactors can be known more accurately by use of Computational Fluid Dynamics (CFD), promoting the development of the relevant reactors.

In some embodiments, a method of collecting the experimental curve of the mass loss percentage of the pyrolysate sample along with change of temperature or time includes:
under a non-isothermal pyrolysis mode, collecting mass loss data corresponding to the pyrolysate sample at different pyrolysis heating rates as sample; and,
under each pyrolysis heating rate, with pyrolysis time and temperature value as input and mass loss percentage as output, obtaining the experimental curve of the mass loss percentage along with change of temperature or time.

In some embodiments, a method of calculating the RMSE of the simulation curve of each mass loss percentage along with change of temperature or time and the experimental curve of the mass loss percentage along with change of temperature or time includes:

$$RMSE ( Ei , Ai ) = \sqrt{\frac{{\sum }_{n = 1}^{N} {( {m}_{exp} ( {t}_{n} ) - {m}_{E , A} ( {t}_{n} ) )}^{2}}{N}}$$

wherein,
i is a sequence number of the kinetics mechanism function in the function library, 1≤i≤I, and I is a total number of the kinetics mechanism functions;
tₙ is the n-th moment in a thermogravimetric experiment process;
n is a moment sequence in the thermogravimetric experiment process, and 1≤n≤N;
N is a total moment number in the thermogravimetric experiment process;
mₑₓₚ(tₙ) is a weight of the pyrolysate sample at the moment tₙ in the thermogravimetric experiment process;
m_{E,A}(tₙ) is a weight of the pyrolysate sample at the moment tₙ in a thermogravimetric simulation process in a case of the optimized activation energy E and the pre-exponential factor A.

In some embodiments, a method of sorting each RMSE and taking the kinetics mechanism function corresponding to the minimum RMSE as the pyrolysis kinetics parameter of the solid material includes:
based on RMSE, sorting all kinetics mechanism functions in a descending order and taking the first-ranked kinetics mechanism function as the most probable mechanism function; and, taking the most probable mechanism function as the pyrolysis kinetics parameter of the solid material.

In some embodiments, the method of obtaining the pyrolysis kinetics parameter of the solid material further includes:

taking the activation energy E corresponding to the most probable mechanism function, the pre-exponential factor A corresponding to the most probable mechanism function and the minimum RMSE as the pyrolysis kinetics parameter of the solid material.

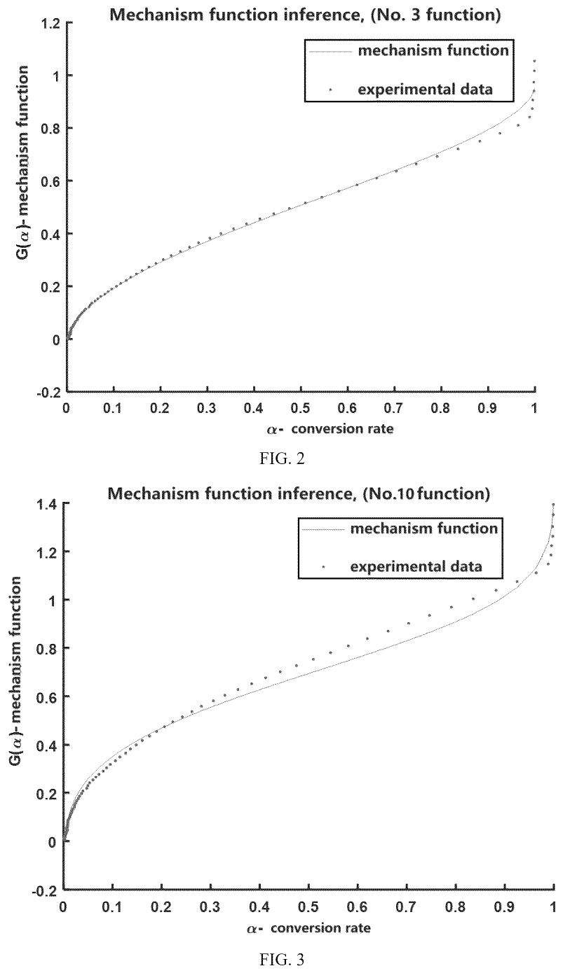

If the No. 3 mechanism function obtained by the method of obtaining the pyrolysis kinetics parameter of the solid material in this embodiment is the most probable mechanism function, the comparison with the experimental data is as shown in FIG. 2, which obviously shows that its RMSE is smaller; the comparison of No. 10 mechanism function of the non-most probable mechanism function and the experimental data is as shown in FIG. 3, which obviously shows that its RMSE is larger.

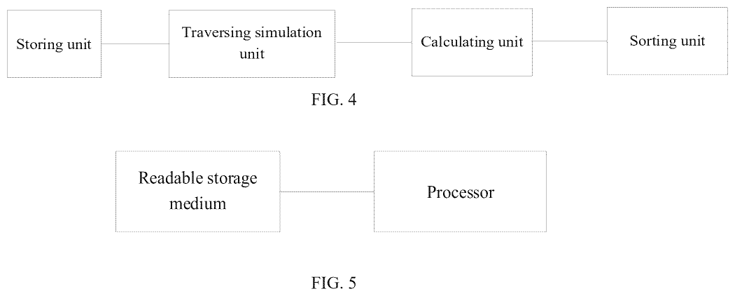

As shown in FIG. 4, some embodiments further provide a system for obtaining a pyrolysis kinetics parameter of a solid material. The system includes at least one computer device. The computer device is configured to include:
a storing unit, configured to store a collected experimental curve of a mass loss percentage of a pyrolysate sample along with change of temperature or time;
a traversing simulation unit, configured to construct a kinetics mechanism function library and traverse each kinetics mechanism function to obtain a simulation curve of the mass loss percentage corresponding to each kinetics mechanism function along with change of temperature or time;
a calculating unit, configured to respectively calculate a root mean square error (RMSE) of the simulation curve of each mass loss percentage along with change of temperature or time and the experimental curve of the mass loss percentage along with change of temperature or time; and,
a sorting unit, configured to sort each RMSE and take the kinetics mechanism function corresponding to a minimum RMSE as the pyrolysis kinetics parameter of the solid material.

The specific functions of the storing unit, the traversing simulation unit, the calculating unit and the sorting unit can be implemented in the computer device.

In some embodiments, the storing unit stores the experimental curve, namely, under a non-isothermal pyrolysis mode, mass loss data corresponding to the pyrolysate sample at different pyrolysis heating rates is collected as sample; and, under each pyrolysis heating rate, with pyrolysis time and temperature value as input and mass loss percentage as output, the experimental curve of the mass loss percentage along with change of temperature or time is obtained and stored.

In some embodiments, the storing unit is further configured to store the kinetics mechanism function library, the simulation curve of the mass loss percentage along with change of temperature or time, the RMSEs and a sorting result of the RMSEs.

In some embodiments, the storing unit can be thought of as one data storage space.

In some embodiments, the traversing simulation unit constructs the kinetics mechanism function library and traverses each kinetics mechanism function to obtain the simulation curve of the mass loss percentage corresponding to each kinetics mechanism function along with change of temperature or time, namely, a method of traversing each kinetics mechanism function to obtain the simulation curve of the mass loss percentage corresponding to each kinetics mechanism function along with change of temperature or time includes: setting an initial activation energy E and an initial pre-exponential factor A; sequentially traversing each kinetics mechanism function and based on quasi-newton method, optimizing the initial activation energy E and the initial pre-exponential factor A at the time of the traversal to obtain the optimized activation energy E and pre-exponential factor A corresponding to each kinetics mechanism function; wherein the optimization aims to minimize the function RMSE(E,A) in the following optimization formula:

$$( {E}_{a + 1} , {A}_{a + 1} ) = ( {E}_{a} , {A}_{a} ) - {Hessian ( RMSE ( {E}_{a} , {A}_{a} ) )}^{- 1} ∇ RMSE ( {E}_{a} , {A}_{a} )$$

wherein,
Eₐ₊₁ represents an activation energy value of the (a+1)-th step of iteration during the optimization process, which is dependent on an activation energy value of the a-th step and gradient information;
Eₐ represents the activation energy value of the a-th step of iteration during the optimization process, and when a=1, an initial activation energy value is obtained;
Aₐ₊₁ represents a pre-exponential factor value of the (a+1)-th step of iteration during the optimization process, which is dependent on a pre-exponential factor value of the a-th step and gradient information;
Aₐ represents the pre-exponential factor value of the a-th step of iteration during the optimization process and when a=1, an initial pre-exponential factor value is obtained;

RMSE (Eₐ, Aₐ) represents an RMSE of the simulation curve and the experimental curve of the mass loss percentage along with change of temperature or time in a case of the activation energy Ea and the pre-exponential factor Aa; and,
with each kinetics mechanism function as thermogravimetric simulation model, using the optimized activation energy E and pre-exponential factor A as parameters of each thermogravimetric simulation model to perform simulation and outputting the simulation curve of the mass loss percentage corresponding to each kinetics mechanism function along with change of temperature or time; wherein,
a is an iteration step number in the optimization process and a≥1.

In some embodiments, the calculating unit respectively calculates a root mean square error (RMSE) of the simulation curve of each mass loss percentage along with change of temperature or time and the experimental curve of the mass loss percentage along with change of temperature or time, namely,
a method of calculating the RMSE of the simulation curve of each mass loss percentage along with change of temperature or time and the experimental curve of the mass loss percentage along with change of temperature or time includes:

$$RMSE ( Ei , Ai ) = \sqrt{\frac{{\sum }_{n = 1}^{N} {( {m}_{exp} ( {t}_{n} ) - {m}_{E , A} ( {t}_{n} ) )}^{2}}{N}}$$

wherein,
i is a sequence number of the kinetics mechanism function in the function library, 1≤i≤I, and I is a total number of the kinetics mechanism functions;
tₙ is the n-th moment in a thermogravimetric experiment process;
n is a moment sequence in the thermogravimetric experiment process, and 1≤n≤N;
N is a total moment number in the thermogravimetric experiment process;
mₑₓₚ(tₙ) is a weight of the pyrolysate sample at the moment tₙ in the thermogravimetric experiment process;
m_{E,A}(tₙ) is a weight of the pyrolysate sample at the moment tₙ in a thermogravimetric simulation process in a case of the optimized activation energy E and pre-exponential factor A.

In some embodiments, the sorting unit sorts each RMSE and takes the kinetics mechanism function corresponding to the minimum RMSE as the pyrolysis kinetics parameter of the solid material, namely,
based on RMSE, all kinetics mechanism functions are sorted in a descending order and the first-ranked kinetics mechanism function is taken as the most probable mechanism function; and
the most probable mechanism function is taken as the pyrolysis kinetics parameter of the solid material.

The electronic device in the embodiments of the present disclosure is described below from the angle of hardware processing.

Some embodiments further provide a computer readable storage medium, storing computer programs/instructions thereon, wherein the computer programs/instructions are executed by a processor to perform any one of the above methods of obtaining the pyrolysis kinetics parameter of the solid material.

Some embodiments further provide a computer device/device/system, including at least one memory, at least one processor and at least one computer program stored on the memory, wherein the computer program is executed by the processor to perform any one of the above methods of obtaining the pyrolysis kinetics parameter of the solid material.

Some embodiments further provide a computer program product including computer programs/instructions, wherein the computer programs/instructions are executed by a processor to perform any one of the above methods of obtaining the pyrolysis kinetics parameter of the solid material.

What is claimed is:

1. A method of obtaining a pyrolysis kinetics parameter of a solid material, comprising:
collecting an experimental curve of a mass loss percentage of a pyrolysate sample along with a change of temperature or time;
constructing a kinetics mechanism function library and traversing each kinetics mechanism function to obtain a simulation curve of the mass loss percentage corresponding to the each kinetics mechanism function along with the change of the temperature or the time;
respectively calculating a root mean square error (RMSE) of the simulation curve of the mass loss percentage corresponding to the each kinetics mechanism function along with the change of the temperature or the time and the experimental curve of the mass loss percentage along with the change of the temperature or the time; and
sorting each RMSE and taking the kinetics mechanism function corresponding to a minimum RMSE as the pyrolysis kinetics parameter of the solid material.

2. The method of claim 1, wherein, a method of collecting the experimental curve of the mass loss percentage of the pyrolysate sample along with the change of the temperature or the time comprises:
under a non-isothermal pyrolysis mode, collecting mass loss data corresponding to the pyrolysate sample at a plurality of pyrolysis heating rates as a sample; and
under each of the plurality of pyrolysis heating rates, with a pyrolysis time and a temperature value as an input and the mass loss percentage as an output, obtaining the experimental curve of the mass loss percentage along with the change of the temperature or the time.

3. The method of claim 1, wherein,
a method of traversing the each kinetics mechanism function to obtain the simulation curve of the mass loss percentage corresponding to the each kinetics mechanism function along with the change of the temperature or the time comprises:
setting an initial activation energy E and an initial pre-exponential factor A;
sequentially traversing the each kinetics mechanism function and based on a quasi-newton method, optimizing the initial activation energy E and the initial pre-exponential factor A at a time of a traversal to obtain an optimized activation energy E and an optimized pre-exponential factor A corresponding to the each kinetics mechanism function; wherein the optimization aims to minimize a function RMSE(E,A) in the following optimization formula:
$$( {E}_{a + 1} , {A}_{a + 1} ) = ( {E}_{a} , {A}_{a} ) - {Hessian ( RMSE ( {E}_{a} , {A}_{a} ) )}^{- 1} ∇ RMSE ( {E}_{a} , {A}_{a} )$$
wherein,
Eₐ₊₁ represents an activation energy value of an (a+1)-th step of an iteration during a optimization process, and the Eₐ₊₁ is dependent on an activation energy value of an a-th step and gradient information;
Eₐ represents the activation energy value of the a-th step of the iteration during the optimization process, and when a=1, an initial activation energy value is obtained;
Aₐ₊₁ represents a pre-exponential factor value of the (a+1)-th step of the iteration during the optimization process, and the Aₐ₊₁ is dependent on a pre-exponential factor value of the a-th step and the gradient information;
Aₐ represents the pre-exponential factor value of the a-th step of the iteration during the optimization process and when a=1, an initial pre-exponential factor value is obtained;
RMSE (Eₐ, Aₐ) represents the RMSE of the simulation curve and the experimental curve of the mass loss percentage along with the change of the temperature or the time in a case of an activation energy Ea and a pre-exponential factor Aa; and
with the each kinetics mechanism function as a thermogravimetric simulation model, using the optimized activation energy E and the optimized pre-exponential factor A as parameters of each thermogravimetric simulation model to perform a simulation and outputting the simulation curve of the mass loss percentage corresponding to the each kinetics mechanism function along with the change of the temperature or the time; wherein,
a is an iteration step number in the optimization process and a≥1.

4. The method of claim 3, wherein,
a method of calculating the RMSE of the simulation curve of the mass loss percentage corresponding to the each kinetics mechanism function along with the change of the temperature or the time and the experimental curve of the mass loss percentage along with the change of the temperature or the time comprises:
$$RMSE ( Ei , Ai ) = \sqrt{\frac{{\sum }_{n = 1}^{N} {( {m}_{exp} ( {t}_{n} ) - {m}_{E , A} ( {t}_{n} ) )}^{2}}{N}}$$
wherein,
i is a sequence number of the kinetics mechanism function in the kinetics mechanism function library, 1≤i≤I, and I is a total number of the kinetics mechanism functions;
tₙ is an n-th moment in a thermogravimetric experiment process;
n is a moment sequence in the thermogravimetric experiment process, and 1≤n≤N;
N is a total moment number in the thermogravimetric experiment process;
mₑₓₚ(tₙ) is a weight of the pyrolysate sample at the n-th moment tₙ in the thermogravimetric experiment process;
m_{E,A}(tₙ) is a weight of the pyrolysate sample at the n-th moment tₙ in a thermogravimetric simulation process in a case of the optimized activation energy E and the optimized pre-exponential factor A.

5. The method of claim 1, wherein,
a method of sorting each RMSE and taking the kinetics mechanism function corresponding to the minimum RMSE as the pyrolysis kinetics parameter of the solid material comprises:
based on the RMSE, sorting all kinetics mechanism functions in a descending order and taking a first-ranked kinetics mechanism function as a most probable mechanism function; and
taking the most probable mechanism function as the pyrolysis kinetics parameter of the solid material.

6. The method of claim 1, further comprising:
taking an activation energy E corresponding to a most probable mechanism function, a pre-exponential factor A corresponding to the most probable mechanism function and the minimum RMSE as the pyrolysis kinetics parameter of the solid material.

7. A system for obtaining a pyrolysis kinetics parameter of a solid material, comprising a computer device, wherein the computer device is configured to comprise:
a storing unit, configured to store a collected experimental curve of a mass loss percentage of a pyrolysate sample along with a change of temperature or time;
a traversing simulation unit, configured to construct a kinetics mechanism function library and traverse each kinetics mechanism function to obtain a simulation curve of the mass loss percentage corresponding to the each kinetics mechanism function along with the change of the temperature or the time;
a calculating unit, configured to respectively calculate an RMSE of the simulation curve of the mass loss percentage corresponding to the each kinetics mechanism function along with the change of the temperature or the time and the experimental curve of the mass loss percentage along with the change of the temperature or the time; and
a sorting unit, configured to sort each RMSE and take the kinetics mechanism function corresponding to a minimum RMSE as the pyrolysis kinetics parameter of the solid material.

8. The system of claim 7, wherein,
the storing unit stores the experimental curve, wherein
under a non-isothermal pyrolysis mode, mass loss data corresponding to the pyrolysate sample at a plurality of pyrolysis heating rates is collected as a sample; and
under each of the plurality of pyrolysis heating rates, with a pyrolysis time and a temperature value as an input and the mass loss percentage as an output, the experimental curve of the mass loss percentage along with the change of the temperature or the time is obtained and stored.

9. The system of claim 7, wherein,
the traversing simulation unit constructs the kinetics mechanism function library and traverses the each kinetics mechanism function to obtain the simulation curve of the mass loss percentage corresponding to the each kinetics mechanism function along with the change of the temperature or the time, wherein
a method of traversing the each kinetics mechanism function to obtain the simulation curve of the mass loss percentage corresponding to the each kinetics mechanism function along with the change of the temperature or the time comprises:
setting an initial activation energy E and an initial pre-exponential factor A;
sequentially traversing the each kinetics mechanism function and based on a quasi-newton method, optimizing the initial activation energy E and the initial pre-exponential factor A at a time of a traversal to obtain an optimized activation energy E and an optimized pre-exponential factor A corresponding to the each kinetics mechanism function; wherein the optimization aims to minimize a function RMSE(E,A) in the following optimization formula:
$$( {E}_{a + 1} , {A}_{a + 1} ) = ( {E}_{a} , {A}_{a} ) - {Hessian ( RMSE ( {E}_{a} , {A}_{a} ) )}^{- 1} ∇ RMSE ( {E}_{a} , {A}_{a} )$$
wherein,
Eₐ₊₁ represents an activation energy value of an (a+1)-th step of an iteration during a optimization process, and the Eₐ₊₁ is dependent on an activation energy value of an a-th step and gradient information;
Eₐ represents the activation energy value of the a-th step of the iteration during the optimization process, and when a=1, an initial activation energy value is obtained;
Aₐ₊₁ represents a pre-exponential factor value of the (a+1)-th step of the iteration during the optimization process, and the Aₐ₊₁ is dependent on a pre-exponential factor value of the a-th step and the gradient information;
Aₐ represents the pre-exponential factor value of the a-th step of the iteration during the optimization process and when a=1, an initial pre-exponential factor value is obtained;
RMSE (Eₐ, Aₐ) represents the RMSE of the simulation curve and the experimental curve of the mass loss percentage along with the change of the temperature or the time in a case of an activation energy Ea and a pre-exponential factor Aa; and
with the each kinetics mechanism function as a thermogravimetric simulation model, using the optimized activation energy E and the optimized pre-exponential factor A as parameters of each thermogravimetric simulation model to perform a simulation and outputting the simulation curve of the mass loss percentage corresponding to the each kinetics mechanism function along with the change of the temperature or the time; wherein,
a is an iteration step number in the optimization process and a≥1.

10. The system of claim 7, wherein,
the calculating unit respectively calculates the RMSE of the simulation curve of the mass loss percentage corresponding to the each kinetics mechanism function along with the change of the temperature or the time and the experimental curve of the mass loss percentage along with the change of the temperature or the time, wherein
a method of calculating the RMSE of the simulation curve of the mass loss percentage corresponding to the each kinetics mechanism function along with the change of the temperature or the time and the experimental curve of the mass loss percentage along with the change of the temperature or the time comprises:
$$RMSE ( Ei , Ai ) = \sqrt{\frac{{\sum }_{n = 1}^{N} {( {m}_{exp} ( {t}_{n} ) - {m}_{E , A} ( {t}_{n} ) )}^{2}}{N}}$$
wherein,
i is a sequence number of the kinetics mechanism function in the kinetics mechanism function library, 1≤i≤I, and I is a total number of the kinetics mechanism functions;
tₙ is an n-th moment in a thermogravimetric experiment process;
n is a moment sequence in the thermogravimetric experiment process, and 1≤n≤N;
N is a total moment number in the thermogravimetric experiment process;
mₑₓₚ(tₙ) is a weight of the pyrolysate sample at the n-th moment tₙ in the thermogravimetric experiment process;
m_{E,A}(tₙ) is a weight of the pyrolysate sample at the n-th moment tₙ in a thermogravimetric simulation process in a case of an optimized activation energy E and an optimized pre-exponential factor A.

11. The system of claim 7, wherein,
the sorting unit sorts each RMSE and takes the kinetics mechanism function corresponding to the minimum RMSE as the pyrolysis kinetics parameter of the solid material, wherein
based on the RMSE, all kinetics mechanism functions are sorted in a descending order and a first-ranked kinetics mechanism function is taken as a most probable mechanism function; and
the most probable mechanism function is taken as the pyrolysis kinetics parameter of the solid material.

12. The system of claim 7, wherein,
the storing unit is further configured to store the kinetics mechanism function library, the simulation curve of the mass loss percentage along with the change of the temperature or the time, the RMSE, and a sorting result of the RMSE.

13. A computer readable storage medium, storing computer programs/instructions thereon, wherein the computer programs/instructions are executed by at least one processor to perform the method of obtaining the pyrolysis kinetics parameter of the solid material of claim 1.

14. A computer device/apparatus/system, comprising at least one memory, at least one processor, and at least one computer program stored on the at least one memory, wherein the at least one processor executes the at least one computer program to perform the method of obtaining the pyrolysis kinetics parameter of the solid material of claim 1.

15. A computer program product, comprising computer programs/instructions, wherein the computer programs/instructions are executed by at least one processor to perform the method of obtaining the pyrolysis kinetics parameter of the solid material of claim 1.
